(19) United States
(12) Patent Application Publication
(10) Pub. No.: US 2008/0200965 A1
(43) Pub. Date: Aug. 21, 2008

(54) Electrically operable incontinence treatment apparatus

(75) Inventor: Peter Forsell, Zug (CH)

Correspondence Address: NIXON & VANDERHYE, PC, 901 NORTH GLEBE ROAD, 11TH FLOOR, ARLINGTON, VA, 22203, US

(73) Assignee: Potencia Medical AG, Zugerstrasse (CH)

(21) Appl. No.: 12/081,545

(22) Filed: Apr. 17, 2008

Related U.S. Application Data
(63) Continuation of application No. 10/543,631, filed on Jul. 28, 2005, now abandoned.

(51) Int. Cl. A61N 1/00

(52) U.S. Cl. 607/41

(57) ABSTRACT

An apparatus for treating an anal or urinary incontinent patient comprises a constriction device applied on the patient's large intestine or urethra, and an electrically powered operation device, which is integrated with the constriction device and adapted to operate the latter to constrict the large intestine or urethra to close the intestinal or urethral lumen and to release the large intestine or urethra to open the intestinal or urethral lumen. An electric power supply is subcutaneously implanted in the patient remote from the constriction device and an insulated electric wire connects the power supply and the electrically powered operation device. The electric wire is resilient and extends helically between the electric power supply and the electrically powered operation device. As a result, the resilient helically extending electric wire is capable of addressing the movements from the large intestine or urethra, because it may easily be temporarily extended without risking breakage.

The present invention relates to an apparatus, for treating an anal or urinary incontinent patient comprising a constriction device adapted to be applied on the patient's large intestine or urethra, and an electrically powered operation device that operates the constriction device to constrict the large intestine or urethra to close the intestinal or urethral lumen and to release the large intestine or urethra to open the intestinal or urethral lumen. The apparatus further comprises an electric power supply adapted to be implanted in the patient remote from the operation device, and an insulated electric wire connecting the electric power supply and the operation device The term “patient” includes an animal or a human being. The term “large intestine” includes the colon, rectum and anal canal.

Anal incontinence is a widespread disease. Several kinds of sphincter plastic surgery are used today to remedy anal incontinence. There is a prior manually operated sphincter system in an initial clinical trial phase where a hydraulic sphincter system connected to an elastic reservoir (balloon) placed in the scrotum is developed. A disadvantage of this system is that thick, hard fibrosis is created around the reservoir by pump movements making the system useless sooner or later.

U.S. Pat. No. 5,593,443 discloses a hydraulic anal sphincter under both reflex and voluntary control. A pressure controlled inflatable artificial sphincter is disclosed in U.S. Pat. No. 4,222,377.

Also urinary incontinence is a widespread problem. Many people are helped through training of the muscles in the pelvic floor but too many have severe problems with urine leakage. Many different solutions to this problem have been tried. For example, there is a prior manually operated urine incontinence treatment apparatus having an artificial hydraulic sphincter device engaging the urethra and connected to an elastic reservoir implanted in the scrotum or in the region of the labia major. A disadvantage of this prior apparatus is that over time hard fibrosis is developed around the reservoir, which may cause malfunction of pumping components. Furthermore, it is a rather complicated task to manually squeeze the elastic implanted reservoir to pump hydraulic fluid to open the sphincter device when the patient needs to urinate. In particular women can get their fingers wet. The created fibrosis will sooner or later become a hard fibroid layer, which may make it even more difficult to pump the reservoir. Yet a further disadvantage is that the use of hydraulic fluid always entails a risk of fluid leaking from implanted hydraulic components.

A prior hydraulic apparatus designed to constrict the urethra is disclosed in U.S. Pat. No. 5,520,606. A prosthetic sphincter with an inflatable cuff, which surrounds the urethra or encloses it on two sides, is disclosed in for example U.S. Pat. Nos. 4,571,749 and 4,222,377. U.S. Pat. No. 4,969,474 discloses a hydraulic method for treating both men and women with urinary incontinence problems in the same way. The apparatus of U.S. Pat. No. 4,969,474 includes a reservoir containing fluid and an inflatable compression means designed to constrict the urethra without risking tissue loss or necrosis to occur. An artificial hydraulically operated urethra sphincter employing an external magnet to achieve closure of the urethra cuff is disclosed in U.S. Pat. No. 5,562,598.

A prior mechanical prosthetic sphincter disclosed in U.S. Pat. No. 4,619,245 comprises a manually controllable actuating component for implanting at a convenient location in the patient's body.

A mechanical apparatus for constricting the large intestine or urethra may typically involve an electrically powered operation device connected to the constriction device. Therefore, such an operation device would be located at the patient's large intestine or urethra. On the other hand, the electric power supply, for example a battery, necessary for powering the operation device with electric energy should be implanted subcutaneously, i.e. remote from the operation device, in order to permit easy access from outside the patient's body for service or replacement of the power supply. In consequence, it is necessary to implant a fairly long electric wire for connecting the power supply to the operation device. However, considering the fact that the apparatus is to be implanted for many years, possibly for the rest of the patient's life, the use of such an electric wire would make the apparatus unreliable, because the movements of the large intestine or urethra might sooner or later break the electric wire.

The object of the present invention is to provide a new reliable electrically operable apparatus for constricting the large intestine or urethra of an incontinent patient.

This object is obtained by an apparatus of the kind presented initially characterised in that the electric wire is resilient and extends helically between the electric power supply and the electrically powered operation device. As a result, the resilient helically extending electric wire is capable of addressing the movements from the large intestine or urethra, because it may easily be temporarily extended without risking breakage.

The electric wire should be designed to conduct a current in the order of milliamperes, which is needed for powering the operation device when operating the constriction device, so that the constriction device exerts a force strong enough to constrict the large intestine or urethra. Thus, the electric wire should have a low resistance.

Advantageously, the apparatus may include an energy transforming device capable of transforming wireless energy transmitted from outside the patient's body into electric energy, whereby the need for regularly replacing the electric power supply, when it is depleted, is eliminated.

Preferably, the operation device includes an electric motor connected to the electric power supply by means of the helical electrical wire. The constriction device may include a hydraulic constriction device inflatable by hydraulic fluid, and the operation device may include an electrically driven pump (for example driven by the electric motor) hydraulically connected to the hydraulic constriction device.

In accordance with a preferred embodiment of the invention, the constriction device is non-inflatable. In this embodiment, the constriction device comprises an elongate constriction member adapted to extend around the large intestine or urethra. The constriction member includes a main portion and two elongated end portions, and the operation device establishes longitudinal relative displacement between the end portions of the constriction member to constrict or release the large intestine or urethra. The operation device may include a movement transferring means in engagement with at least one of the end portions of the constriction member and operable by the electric motor to displace the one end portion relative to the other end portion of the constriction member. The movement transferring means may include a gear wheel fixed to the other end portion of the constriction member and a gear rack formed on the one end portion of the constriction member, the gear wheel and the gear rack being in mesh with each other. The operation device may further include a worm gear connected between the electric motor and the gear wheel.

Generally, the constriction device includes a housing containing the operation device, and the elongate constriction member and the housing are adapted to form a loop around the patient's large intestine or urethra.

Advantageously, the apparatus includes a control device, suitably in the form of a wireless remote control, for controlling the operation device to operate the constriction device. The control device may include an internal, preferably programmable, control unit implantable in the patient for controlling the operation device. Furthermore, the control device may include an external control unit outside the patient's body, wherein the internal control unit is programmable by the external control unit.

The apparatus may include at least one implantable sensor for sensing at least one physical parameter of the patient. The sensor may be a pressure sensor for directly or indirectly sensing the pressure against the constriction device. The control device may suitably control the operation device in response to signals from the sensor. Where the control device includes an internal control unit, the internal control unit may control the operation device in response to signals from the sensor.

The apparatus may include a switch implantable in the patient for directly or indirectly switching the electric energy supplied by the electric power supply.

In the enclosed drawings:

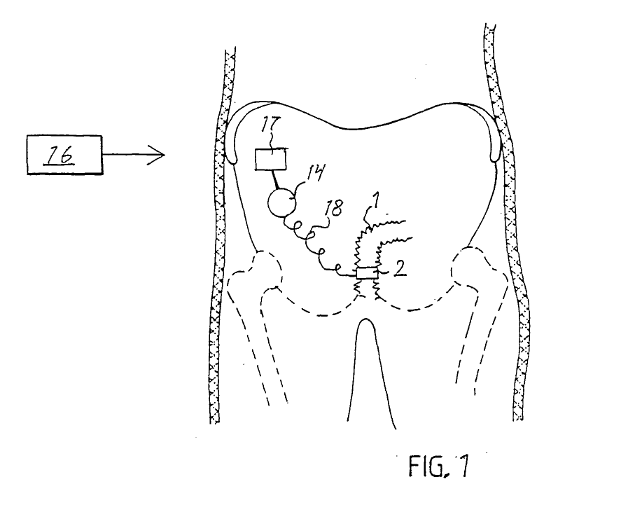

FIG. 1 is a schematic view of an apparatus according to the present invention implanted in an incontinent patient, and

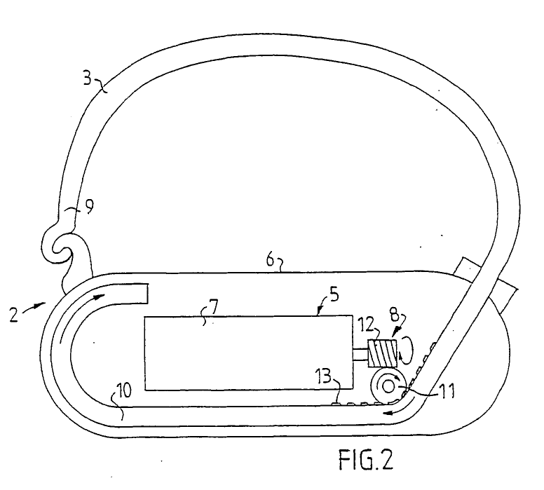

FIG. 2 schematically illustrates an embodiment of a constriction device of the apparatus for use in the embodiment of FIG. 1.

FIG. 1 shows an apparatus of the present invention for treating an anal incontinent patient comprising a constriction device 2 applied on the rectum 1. The constriction device 2 includes an elongate operable constriction member 3 extending in a loop around and constricting the rectum 1 to normally close the rectal lumen. An electrically powered operation device 5 that operates the constriction member 3 to open and close the rectal lumen is housed in an elongate housing 6, see FIG. 2. The constriction member 3 has a first end portion 9 releasably connected to the housing 6 and a second end portion 10 connected to the operation device 5. The operation device 5 includes an electric motor 7 and a movement transferring means 8 in engagement with the end portion 10. The electric motor 7 operates the movement transferring means 8 to displace the end portion 10 relative to portion 9 in the loop formed by the constriction member 3. The movement transferring means 8 includes a gear wheel 11 fixed to the housing 6, a worm gear 12 connected between the electric motor 7 and the gear wheel 11, and a gear rack 13 formed on the end portion 10, wherein the gear wheel 11 and the gear rack 13 are in mesh with each other.

A rechargeable electric power supply 14 is subcutaneously implanted in the patient. An external remote control 16 controls the operation device 5 and transmits signals that are received by a combined control and energy transforming unit 17 subcutaneously implanted in the patient. The unit 17 is electrically connected to the electric power supply 14 and transforms the energy of the signals into an electric current that is used for charging the electric power supply 14. For example, the signals may include electromagnetic waves and the unit 17 may include an electric p-n junction element that transforms the wireless energy into an electric current.

A resilient insulated electric wire 18 connects the power supply 14 and the electric motor 7. The electric wire 18 extends helically between the power supply 14 and housing 6 that contains the motor 7, in order to permit the electric wire 18 to be temporarily extended when movements of the rectum occur, so that the risk of breaking the electric wire 18 is eliminated.

1. An apparatus for treating an anal or urinary incontinent patient comprises a constriction device adapted to be applied on the patient's large intestine or urethra, an electrically powered operation device that operates the constriction device to constrict the large intestine or urethra to close the intestinal or urethral lumen and to release the large intestine or urethra to open the intestinal or urethral lumen, an electric power supply adapted to be implanted in the patient remote from the operation device, and an insulated electric wire power connecting the electric power supply and the operation device, the electric power wire being resilient and extending helically between the electric power supply and the electrically powered operation device.

2. The apparatus according to claim 1, further comprising an implantable energy transforming device capable of transforming wireless energy transmitted from outside the patient's body into electric energy.

3. The apparatus according to claim 1, wherein the operation device comprises an electric motor connected to the electric power supply by means of the helical electrical wire.

4. The apparatus according to claim 3, wherein the constriction device is non-inflatable.

5. The apparatus according to claim 4, wherein the constriction device comprises an elongate constriction member adapted to extend around the large intestine or urethra.

6. The apparatus according to claim 5, wherein the constriction member comprises a main portion and two elongated end portions, and the operation device establishes longitudinal relative displacement between the end portions of the constriction member.

7. The apparatus according to claim 6, wherein the operation device comprises an electric motor and a movement transferring means in engagement with at least one of the end portions of the constriction member and operable by the electric motor to displace said one end portion relative to the other end portion of the constriction member.

8. The apparatus according to claim 7, wherein the movement transferring means comprises a gear wheel fixed relative to said other end portion of the constriction member and a gear rack formed on said one end portion of the constriction member, the gear wheel and the gear rack being in mesh with each other.

9. The apparatus according to claim 8, wherein the operation device comprises a worm gear connected between the electric motor and the gear wheel.

10. The apparatus according to claim 1, wherein the constriction device comprises a hydraulic constriction device and the operation device comprises an electrically driven pump hydraulically connected to the hydraulic constriction device.

11. The apparatus according to claim 5, wherein the constriction device comprises a housing containing the operation device, and the elongate constriction member and the housing are adapted to form a loop around the patient's large intestine or urethra.

12. The apparatus according to claim 1, further comprising a wireless remote control for controlling the operation device to operate the constriction device.

13. The apparatus according to claim 1, further comprising at least one implantable sensor for sensing at least one physical parameter of the patient.

14. The apparatus according to claim 13, wherein the sensor comprises a pressure sensor for directly or indirectly sensing the pressure against the constriction device.

15. The apparatus according to claim 14, further comprising a control device for controlling the operation device in response to signals from the sensor.

16. The apparatus according to claim 15, wherein the control device comprises an internal control unit implantable in the patient for controlling the operation device in response to signals from the sensor.

17. The apparatus according to claim 1, further comprising a control device for controlling the operation device

18. The apparatus according to claim 17, wherein the control device comprises an internal control unit implantable in the patient for controlling the operation device.

19. The apparatus according to claim 18, wherein the internal control unit is programmable.

20. The apparatus according to claim 19, wherein the control device comprises an external control unit outside the patient's body, the internal control unit being programmable by the external control unit.

21. The apparatus according to claim 1, further comprising a switch implantable in the patient for directly or indirectly switching the electric energy supplied by the electric power supply.
